(19) United States
(12) Patent Application Publication
(10) Pub. No.: US 2024/0257960 A1
(43) Pub. Date: Aug. 1, 2024

(54) INSTRUMENT SELECTION OPTIMIZATION

(71) Applicant: Fortive Corporation, Everett, WA (US)

(72) Inventors: Suneel Shorey, Newport Beach, CA (US); Jason Rawlings, Murfreesboro, TN (US); Krishna Yalamanchili, Cornelius, NC (US); Geoffrey Morgan, Pittsburgh, PA (US); Jie Zhao, McMinnville, TN (US); Darshana Mukundan, Pittsburgh, PA (US); Aaron Acton, McKees Rocks, PA (US)

(21) Appl. No.: 18/424,389

(22) Filed: Jan. 26, 2024

Related U.S. Application Data
(60) Provisional application No. 63/441,560, filed on Jan. 27, 2023.

(51) Int. Cl. G16H 40/40; G16H 30/40; G16H 40/20

(52) U.S. Cl. CPC G16H 40/40; G16H 30/40; G16H 40/20

(57) ABSTRACT

In various embodiments, a process for instrument selection optimization includes receiving a specification of surgical implements available for use during a surgical operation, receiving an image depicting at least a portion of the available surgical implements utilized during the surgical operation, and at least in part automatically generating a record of the utilized surgical implements depicted in the image. The process includes storing the record in a data storage of surgical implement utilization data, and automatically analyzing records stored in the data storage of surgical implement utilization data to identify one or more surgical implement preparation groupings optimizing one or more specified utilization metrics.

### CROSS REFERENCE TO OTHER APPLICATIONS

This application claims priority to U.S. Provisional Patent Application No. 63/441,560 entitled INSTRUMENT SELECTION OPTIMIZATION filed Jan. 27, 2023 which is incorporated herein by reference for all purposes.

### BACKGROUND OF THE INVENTION

Surgical procedures (or more generally, medical procedures) are typically performed by medical practitioners (e.g., surgeons) using surgical implements (e.g., instruments) that are prepared and placed in surgical trays for use during the medical procedure/surgery. The selection of implements and/or composition of surgical trays affects the cost, duration, and efficiency with which the procedure is performed and impacts the overall profitability of a hospital. Surgical implement usage varies by surgeon and the surgical procedure being undertaken. In one aspect, implements that are not used during a procedure are processed for re-use after every procedure and repaired after a certain number of reprocessing cycles, which involves additional cost and time. Other issues from overloaded trays include: increased operating room (OR) turnover time, longer surgical procedure time, and more case delays resulting from SPD-related issues. In another aspect, implements tend to be added to existing trays and with the addition of new procedures and technologies, additional trays with new implements are added over time, which could lead to increased waste. Thus, there is a need for optimized implement selection.

### BRIEF DESCRIPTION OF THE DRA WINGS

Various embodiments of the invention are disclosed in the following detailed description and the accompanying drawings.

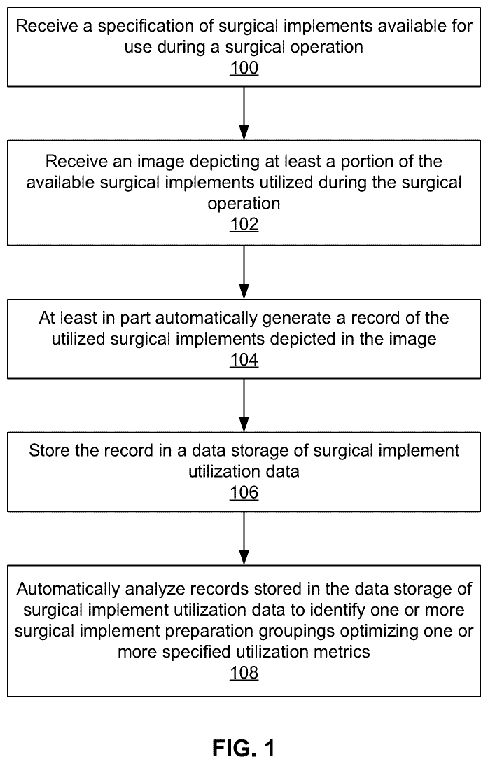

FIG. 1 is a flow diagram illustrating an embodiment of a process for instrument selection optimization.

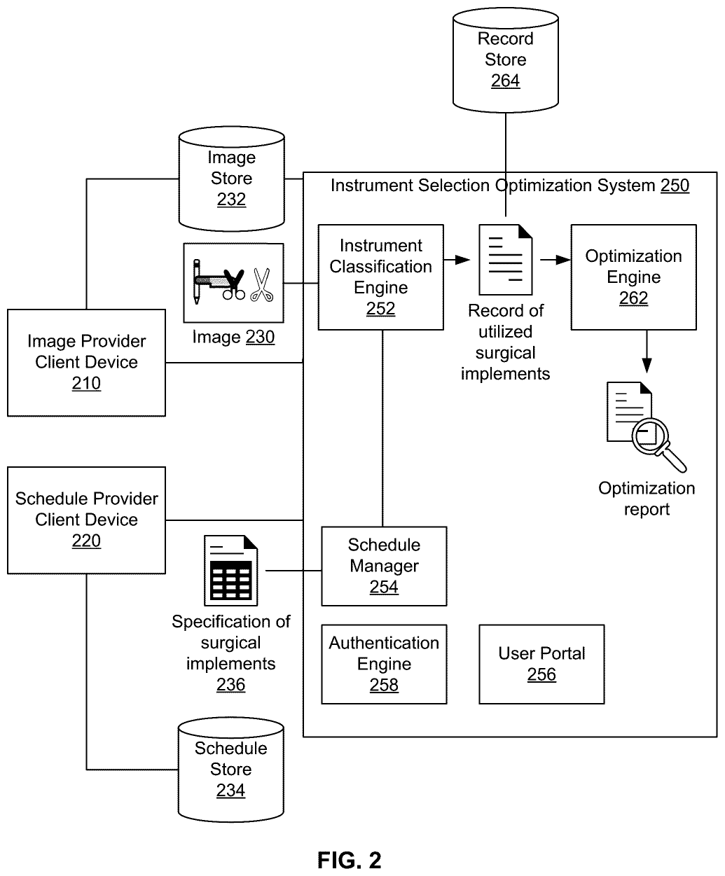

FIG. 2 is a block diagram illustrating an embodiment of a system for instrument selection optimization.

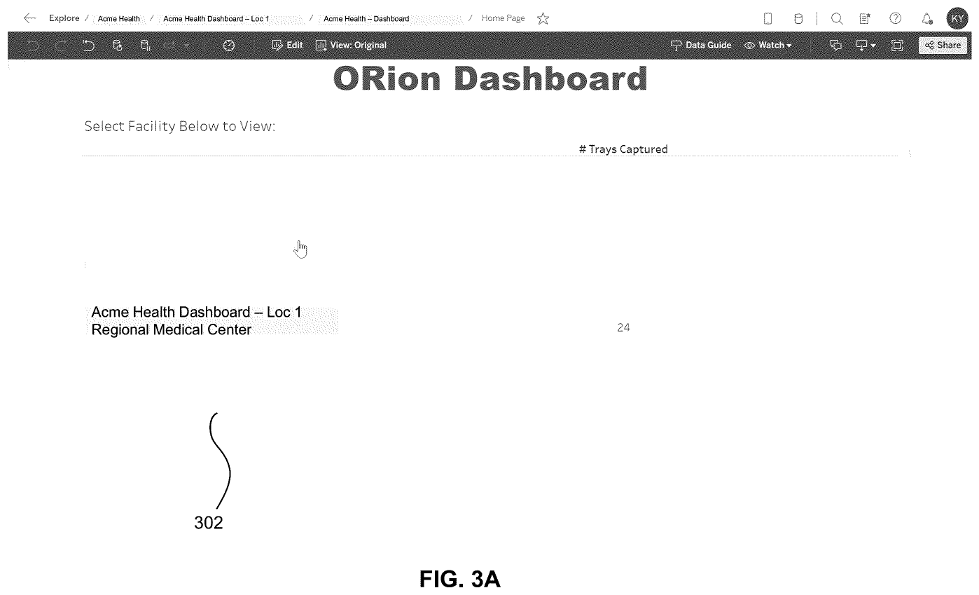

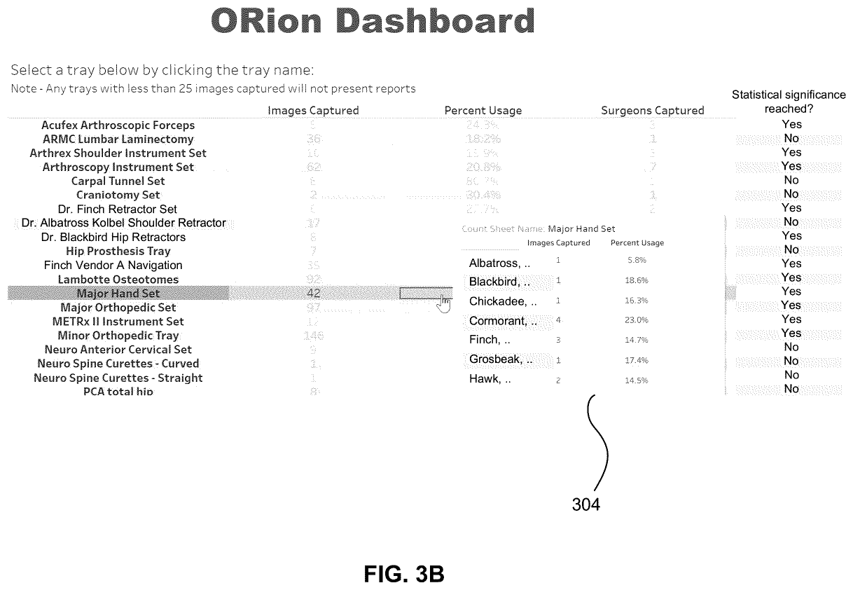

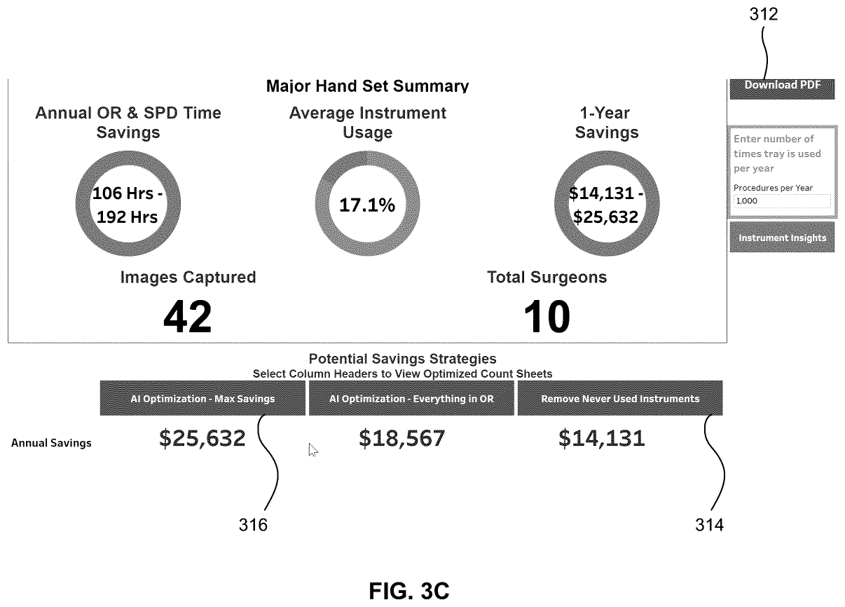

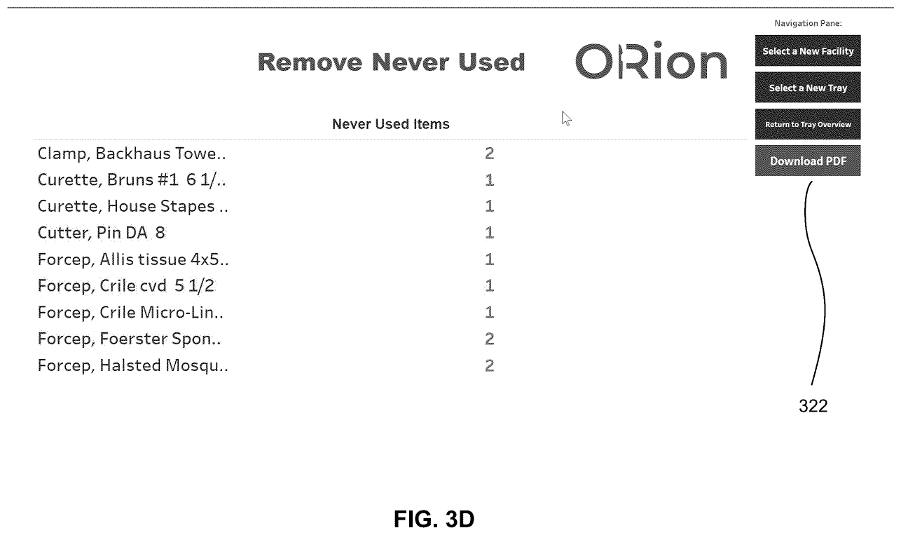

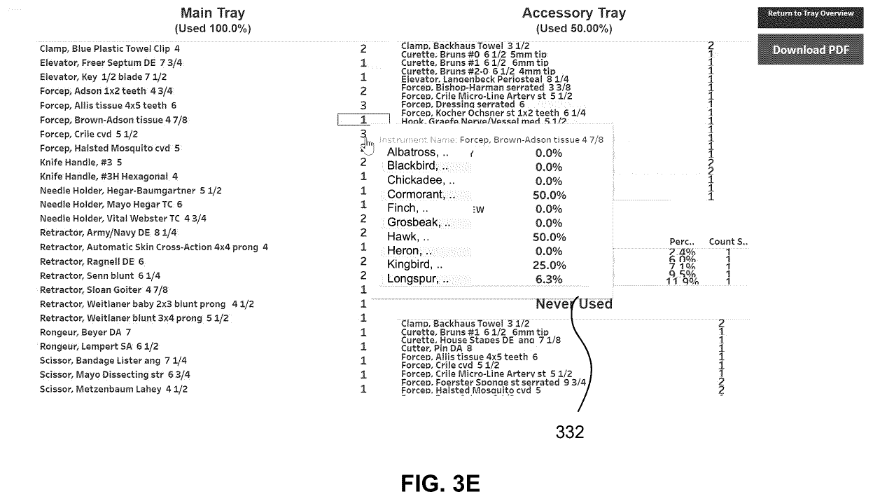

FIG. 3A shows an example of a user interface for instrument selection optimization.

FIG. 3B shows an example of a user interface for instrument selection optimization including tray analysis.

FIG. 3C shows an example of a user interface for instrument selection optimization including optimization information for a particular tray.

FIG. 3D shows an example of a user interface for instrument selection optimization including never used instruments.

FIG. 3E shows an example of a user interface for instrument selection optimization including an optimization for a particular tray.

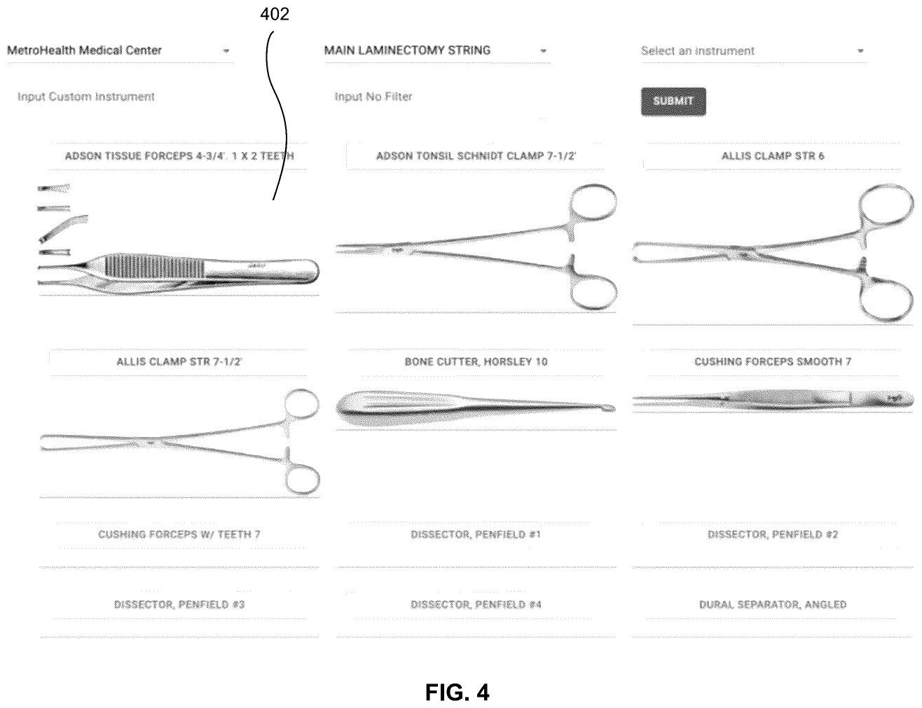

FIG. 4 shows an example of a user interface for an annotation engine.

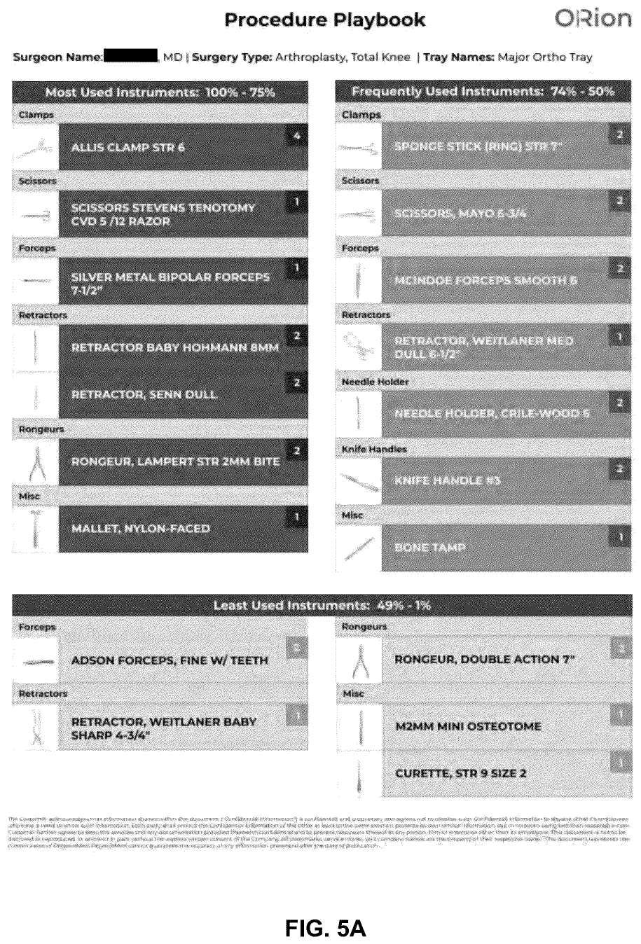

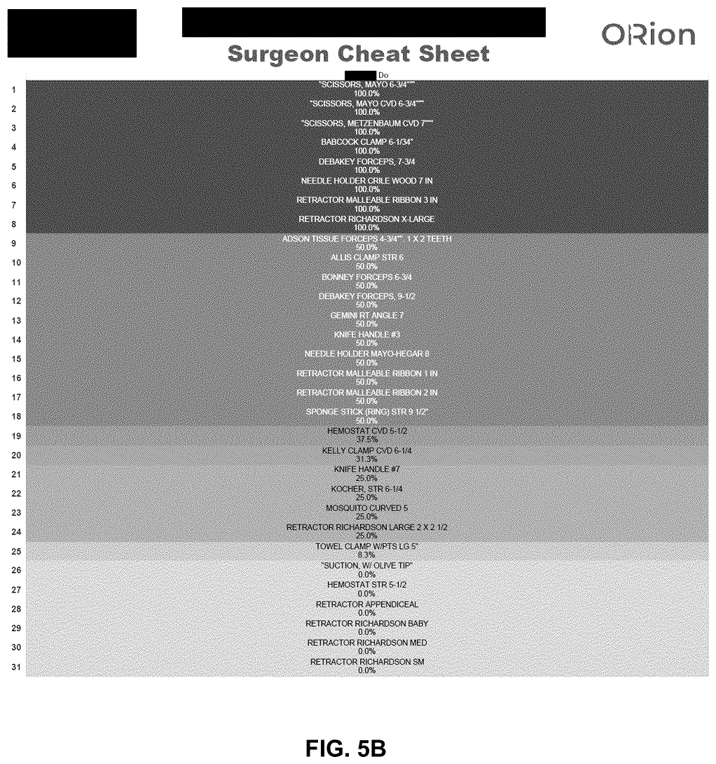

FIG. 5A shows an example of a report including a procedural playbook.

FIG. 5B shows an example of a report including a procedural playbook.

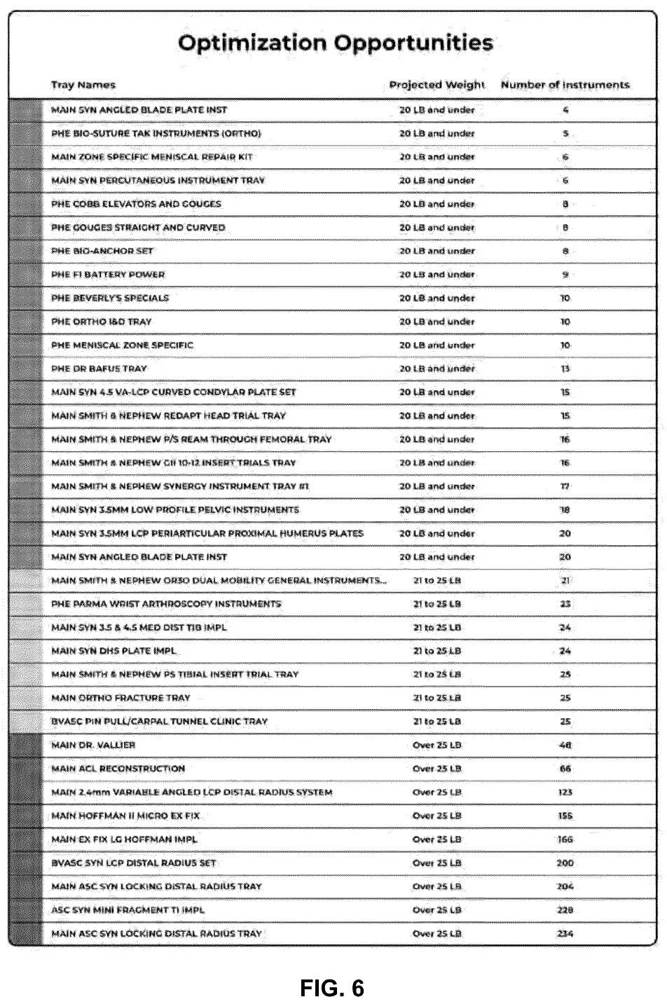

FIG. 6 shows an example of a report including a tray compliance roadmap. In various embodiments, the roadmap can be used to meet weight limits of trays and reduce workplace injuries.

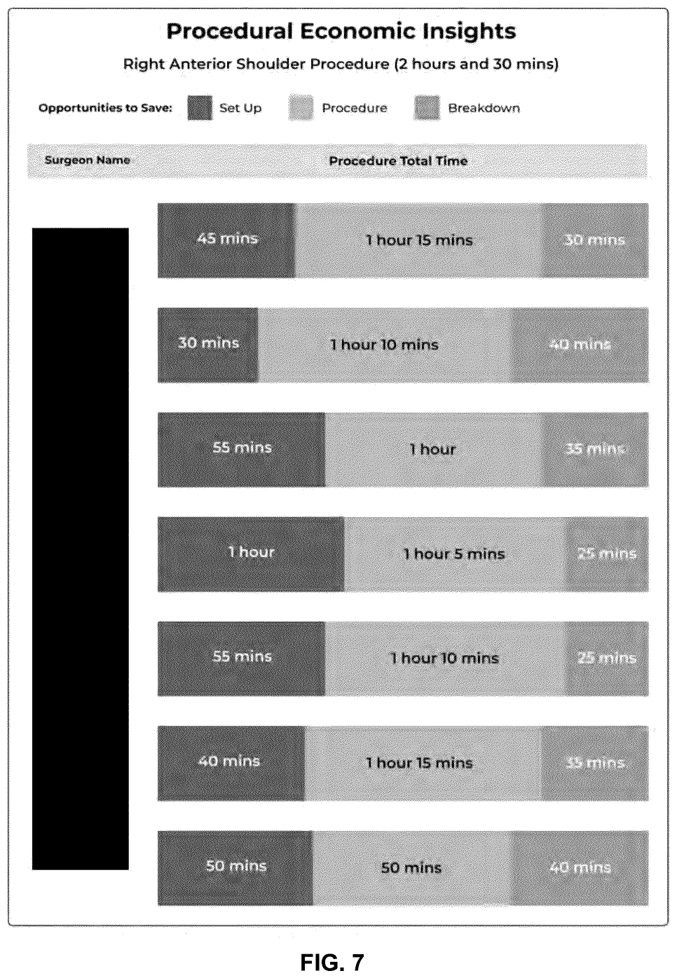

FIG. 7 shows an example of a report including procedural economic insights. The insights may help establish best practices and generate surgeon report cards.

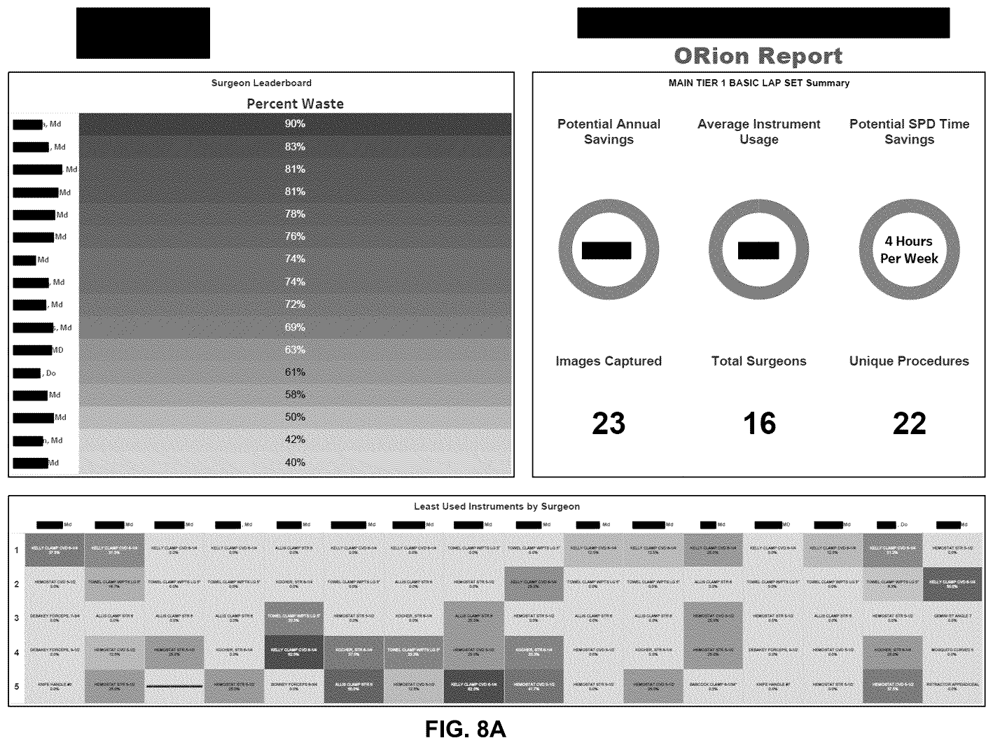

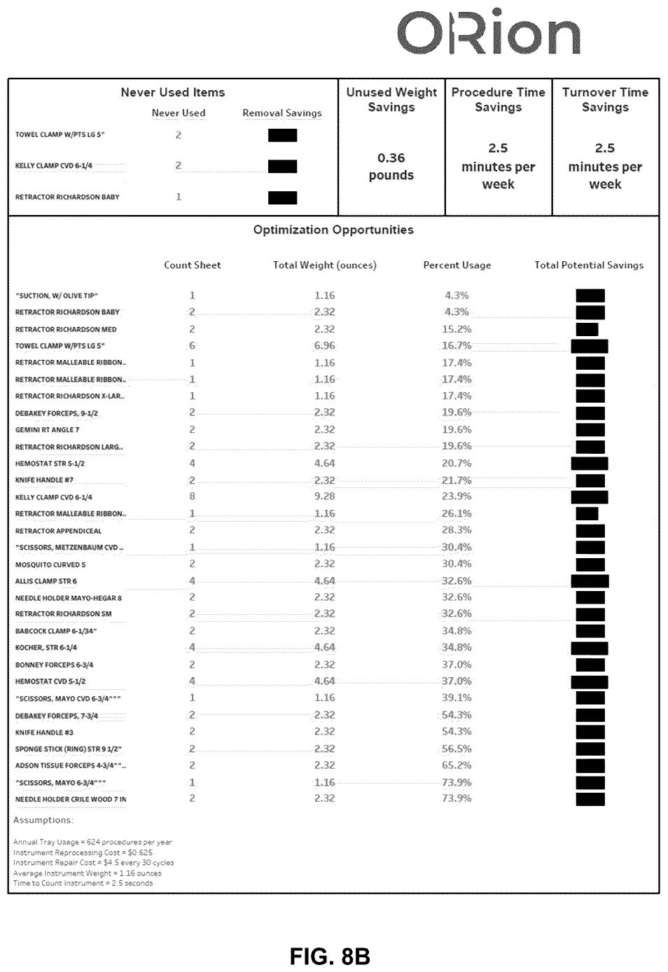

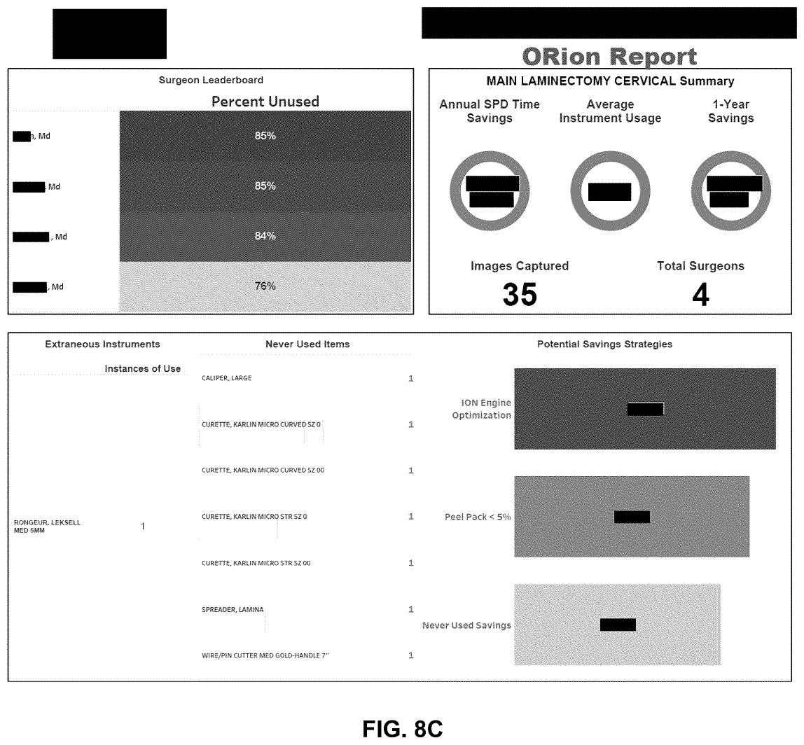

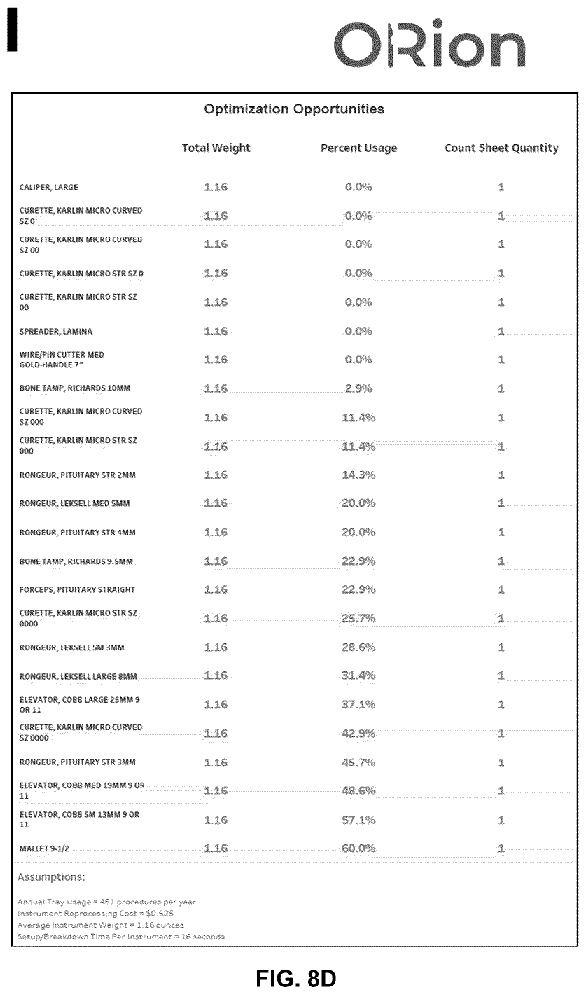

FIG. 8A shows an example of a report including service line optimization.

FIG. 8B shows an example of a report including service line optimization.

FIG. 8C shows an example of a report including service line optimization.

FIG. 8D shows an example of a report including service line optimization.

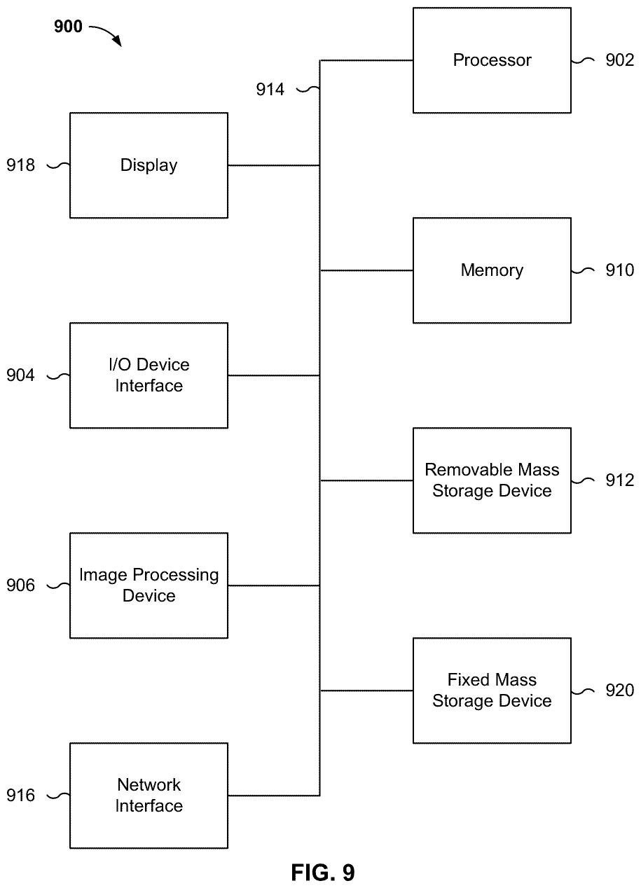

FIG. 9 is a functional diagram illustrating a programmed computer system for instrument selection optimization in accordance with some embodiments.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

It was observed that during the majority of medical procedures, many implements are consistently unused, duplicated across trays in the same surgical procedure, presented in an inefficient manner, among other factors, leading to waste. Usage rates may vary due to factors such as the particular surgeon performing the procedure, the type of procedure, the complexity or the case, patient health, etc. The disclosed techniques determine and optimize implement selection. For example, surgical trays (groupings of implements) may be automatically/programmatically determined to optimize implements selection and use. In various embodiments, instruments (or more generally, any item of inventory related to a medical procedure such as tools or soft goods like sponges or sutures) can be selected for placement in a surgical tray. Given sufficient data, the optimal selection can be automatically/programmatically determined. One or more groups of implements (e.g., a group corresponds to a surgical tray) can be formed, each tray including one or more implements. The optimized composition of the trays can be output in a report, for example.

The disclosed techniques find application in a variety of settings. Although primarily described using the example of surgical instruments, the disclosed techniques may be applied to various implements including other inventory used for surgery, medical procedures, or other processes.

FIG. 1 is a flow diagram illustrating an embodiment of a process for instrument selection optimization. This process may be implemented on or by an instrument selection optimization such as the system 250 shown in FIG. 2 or a processor such as processor 902 shown in FIG. 9.

In the example shown, the process begins by receiving a specification of surgical implements available for use during a surgical operation (100). The specification of surgical implements available for use during a surgical operation includes an inventory of surgical implements that may be used during the operation. The specification may vary from surgeon to surgeon, hospital to hospital, healthcare system to healthcare system, etc.

An example of a specification of surgical implements available for use during a surgical operation is an instrument count sheet. An instrument count sheet includes a bill of materials for the composition of a specific surgical tray, which may also be specific to a surgeon. The instrument count sheet can be exported in multiple formats e.g., via a CSV, PDF, Excel file, or from a hand written document. The format of the instrument count sheets can vary across sources and sites of care (e.g., hospitals, ambulatory surgery centers, or vendor-owned surgical trays).

The process receives an image depicting at least a portion of the available surgical implements utilized during the surgical operation (102). The image may include a visual representation showing a shape/form of the implements. The implements may be provided in a tray. For example, the image includes a photograph of a surgical tray. The photograph may include one or more surgical trays that include one or more instruments. The image can be captured by any device with a suitable megapixel rated camera, such as a smartphone. The image can be captured by a technician or other user after instruments have been used (e.g., at the end of a medical procedure).

In various embodiments, the image includes only those instruments that have been used (e.g., opened). The un-used instruments are kept out of sight for example by being placed beneath a cloth or otherwise removed from view of the camera. Alternatively, the image may include a mixture of used and un-used instruments and subsequent image processing classifies those instruments that have been used vs. un-used.

The image may be accompanied by metadata. For example, the user who captured the image may indicate what type of tray or content is in the image. Alternatively, the user does not need to provide any information and the information is automatically determined when processing the image, as further described herein.

The process at least in part automatically generates a record of the utilized surgical implements depicted in the image (104). The record may include an identification of the types or other characteristics of the utilized surgical implements depicted in the image. For example, surgical implements may be labeled using image recognition, manual annotation, machine learning annotation, or the like.

In various embodiments, automatically generating the record of the utilized surgical implements depicted in the image includes annotating each of at least a subset of the utilized surgical implements based at least on a plurality of previously received images. For example, an annotation engine is used to identify instruments that were used in the procedure from a specific surgical tray and automatically/programmatically creates a known set of data. The identification can be based on images as further described herein, and/or the instrument classifications.

The annotation engine includes a trained machine learning model, may solicit input from a human annotator, or be a hybrid of both. The annotation engine can include a machine learning model which uses data from a human annotator and/or a separate training set of data compiled from thousands of instrument images in order to learn how to automatically identify instruments correctly in conjunction with the annotator. An example of an annotation engine is instrument classification engine 252, further described with respect to FIG. 2. A user interface for a human annotator may be provided via user portal 256. An example of a user interface for an annotation engine is shown in FIG. 4.

Data may be consolidated over one or more procedures to obtain a statistically significant number of images. For example, data is continually captured for instrument usage from a specific surgical tray until a statistically significant sample size is achieved. A robustness analysis algorithm is used to determine when a statistically significant sample size has been collected.

In various embodiments, a total number of the plurality of previously received images utilized for the annotation is based at least on a robustness analysis of the plurality of previously received images performed using a threshold indicating a reliability of the plurality of the previously received images. For example, the process performs the robustness analyzing including by subsampling at random from a set of images. Suppose there were 50 pictures taken of a particular tray. The robustness analysis determines how reliable the results would be if only a subset of the pictures is used, for example, if only 40 pictures vs. 35 vs. 20 pictures are used. Identifying the smallest subset size for which results are sufficiently reliable decreases processing resources used and avoids over-training associated machine learning models.

In various embodiments, wherein a total number of the plurality of previously received images utilized for the annotation is based at least on a coverage analysis of the plurality of previously received images performed using a threshold indicating a level of representation of the surgical operation with respect to a group of surgical operations. The level of representation is a quantification of whether the surgical operation (e.g., a particular surgical operation) is sufficiently represented when considering a group of surgical operations. The coverage analysis may answer questions such as: is there sufficient representation of a particular surgeon whose team tends not to provide input (e.g., not capture images or otherwise participate in providing input data), or is there sufficient representation of this surgical operation? A visualization of the robustness analysis and/or coverage analysis may be generated and output via a user interface such as via user portal 256.

In various embodiments, automatically generating the record of the utilized surgical implements depicted in the image includes identifying each of at least a subset of the utilized surgical implements without using the specification of the surgical implements available for use during the surgical operation. Alternatively, the record of the utilized surgical implements depicted in the image is based at least on the specification of the surgical implements available for use during the surgical operation. For example, the surgical implements depicted in the image are classified using the specification of surgical implements available for use during a surgical operation received at 100. The image may include instruments that were not on the specification of surgical implements (possibly from another tray or a single use Peel Pack). These instruments may also be annotated.

As further described herein, metadata may be collected with or identified from the images. For example, manufacturer/vendor data may be detected and analyzed to provide market insight and analytics.

The process stores the record in a data storage of surgical implement utilization data (106). The record may be stored and retrieved from the data storage for analysis. Referring briefly to FIG. 2, the record may be stored in record store 264. For example, once a statistically significant sample size (e.g., images) is achieved, the data is then compiled and stored. Returning to FIG. 1, the compiled data may be used by the process to interpret actual instrument usage data. The record may be used for optimizing one or more specified utilization metrics by identifying a surgical implement preparation grouping as follows.

The process automatically analyzes records stored in the data storage of surgical implement utilization data to identify one or more surgical implement preparation groupings optimizing one or more specified utilization metrics (108). The records can be compiled/analyzed (e.g., by a data science team or programmatically/automatically) to determine metrics such as corresponding usage rate for each instrument. In various embodiments, suggestions on how to optimize trays, service lines, complete inventory, or the like may be determined and provided.

In various embodiments, the automatic analysis includes performing tray analysis. Tray analysis refers to analysis associated with the configuration of implements on a tray (also called “tray configuration”). Optimizations may be identified for how to optimize a particular tray setup such as which instruments to include in a specific tray. For example, the one or more surgical implement preparation groupings are organized into one or more trays. The one or more trays includes a core tray including a first subset of the utilized surgical implements and an accessory tray including a second subset of the utilized surgical implements. Optimizations may be determined for how to optimize multiple trays used in a procedure. Instruments may be classified by ownership such as hospital-owned vs. vendor-owned (on consignment). Vendor-owned trays may be optimized separately from hospital-owned trays, for example.

In various embodiments, the automatic analysis includes determining a co-usage pattern of a first one of the one or more surgical implements that is used with a second one of the one or more surgical implements at a frequency that meets a usage threshold. The co-usage pattern refers to instruments that tend to be used together or not used together. Identifying a co-usage pattern allows various optimization suggestions to be made. For example, a co-usage pattern allows a single tray to be split into two trays: a core tray (those that are used together) and a non-core (“accessory”) tray (less frequently used and not with each other).

In various embodiments, the automatic analysis includes performing service-line analysis. That is, the automatic analysis includes identifying optimizations for (full) service line inventories of trays that exist in hospitals. This may be helpful for standardizing procedures across hospitals within a particular system. For example, when a healthcare network acquires a new hospital, there may be options for optimizations. A user of the instrument selection optimization system may indicate desired parameters such as the optimal number of trays when factoring in the addition of the new hospital. Best practices may be suggested or adjusted accordingly.

The one or more specified utilization metrics includes at least one of: usage rate for a particular instrument, usage rate for a particular surgeon, and/or usage rate for a particular procedure. In various embodiments, the utilization metrics may be specified and adjusted via an application programming interface (API) and/or a user interface. For example, constraints (such as the number of peel packs, per surgeon) are presented on the dashboard as a slider. A user can adjust the slider to provide their input, which causes the process to include/update this metric and determine an optimization that takes this into consideration. As another example, a user may query for an optimization by a specified number of surgeons, a specified number of procedures, as a function of the number of procedures, how many trays the service line has, etc. As yet another example, a user may specify “I want 20 to 30 instruments. What are the estimated cost/benefits from the best version of each of those options?”

In various embodiments, the automatic analysis includes performing a robustness analysis to determine a reliability of the identified one or more surgical implement preparation groupings. The robustness analysis is the same as the example described with respect to 104 unless otherwise described herein. Robustness analysis refers to determining a volume of data needed to reach statistical significance. A minimum subsample size is determined, where using the minimum subsample size results in the performance meeting a threshold (e.g., achieving sufficient confidence).

In various embodiments, the automatic analysis includes performing a coverage analysis to determine a representation level of a characteristic associated with the surgical operation. The coverage analysis is the same as the example described with respect to 104 unless otherwise described herein. Coverage analysis refers to determining gaps in the data used for analysis. For example, whether a particular surgeon or procedure has sufficient representation in the data to obtain a result that would be applicable to that particular surgeon or procedure. The coverage analysis may be performed based at least on the specification of surgical implements available for use during a surgical operation. The specification of surgical implements may be used to identify what implements are under- vs. over-represented.

Referring briefly to FIG. 2, the utilization metrics may be specified by various users, such as an instrument selection optimization system 250, a schedule provider 220, an image provider 210 (e.g., a hospital that provides input data), or others. For example, the departure of a surgeon may be taken into account by allowing this to be indicated and removing corresponding data, e.g., during a coverage analysis phase.

Example optimizations include but are not limited to:
· remove never-used instruments,
create a main tray with most-used instruments,
reduce a number of instruments (e.g., from 100 to 50 instruments; the remaining 50 instruments are provided in an accessory tray),
predict a frequency of using/opening an instrument,
adjust accessory trays such as the number of accessory trays,
optimize a size of a main tray (e.g., number of instruments to include in a main tray), and
optimize a set of instruments to be kept in an operating room that caters to all surgeons who use that room.

In various embodiments, the process outputs a report based at least on the automatic analysis of the records stored in the data storage of surgical implement utilization data to identify the one or more surgical implement preparation groupings optimizing the one or more specified utilization metrics. Examples of reports are further described with respect to FIGS. 5A to 8D.

FIG. 2 is a block diagram illustrating an embodiment of a system for instrument selection optimization. The system includes an instrument selection optimization system 250, an image provider client device 210, an image store 232, a schedule provider client device 220, and a schedule store 234. Although depicted as external to/remote from the instrument selection optimization system 250, the image store 232, schedule store 234, and record store 264 may instead be locally hosted by the instrument selection optimization system 250. The data of the stores 232, 234, and 264 may be stored by various devices or combinations of devices.

The image provider client device 210 is configured to capture and/or provide images depicting at least a portion of available surgical implements utilized during the surgical operation. The images may be processed using the disclosed techniques, e.g., the process of FIG. 1, to identify one or more surgical implement preparation groupings optimizing one or more specified utilization metrics.

The image store 232 is configured to store images such as those captured by the image provider client device 210. Alternatively, images may be directly sent to the instrument selection optimization system 250 without first being stored in the image store 232. The image store 232 is communicatively coupled to the image provider client device 210 and the instrument selection optimization system 250.

The schedule provider client device 220 is configured to collect and provide a specification of surgical implements available for use during a surgical operation. The specification of surgical implements is sometimes referred to as “a schedule.” The schedule provider client device may include or be included in an electronic health record (EHR) or electronic medical record (EMR) system.

The schedule store 234 is configured to store schedules or specifications of surgical implements available for use during a surgical operation such as those collected by the schedule provider client device 220. The schedule store 234 is communicatively coupled to the image provider client device 210 and the instrument selection optimization system 250. As shown in this example, in various embodiments, the specification of surgical implements 236 available for use during a surgical operation is received from a first source (here, the image provider client device 210) and the image 230 depicting at least a portion of the available surgical implements utilized during the surgical operation is received from a second source (here, the schedule provider client device 220) different from the first source.

The client device 210 and 220 may include any processing device such as a mobile smartphone, tablet, computer, or the like. An example of a processing device is further described herein with respect to FIG. 9. The client device may be customized for purposes of instrument selection optimization, e.g., loaded with an appropriate software application programmed to perform the disclosed techniques. Functionalities of the device such as those that may impact negatively security or are otherwise unnecessary may be disabled.

In various embodiments, the client device 210 sits in the operating rooms and snaps pictures for the data scientists to use. Images are captured in the field, e.g., by a surgical technician. Metadata may be input or otherwise associated with the images. Examples of metadata include the operating room, the facility, the surgeon, the type of surgical procedure, etc. The images may be annotated (manually and/or by a machine), and then associated with the surgery information. The surgery information may be provided asynchronously from the image capture. For example, the surgery information may be uploaded by hospitals periodically such as weekly. The surgery information may be provided in various formats such as a CSV or Excel file.

In various embodiments, the client device may be dynamically reconfigured. For example, the client device may be reconfigured based on a particular server environment. As another example, a tenant ID may be provided to provide a separate, isolated tenant. The user may provide input such as selection of a server, facility, or operating room and the client device would be configured to be appropriate for that environment. Tray options may vary from facility to facility, and the appropriate options are displayed. Adaptations are made for a client device's abilities, hardware and software characteristics.

The instrument selection optimization system 250 includes an instrument classification engine 252, an optimization engine 262, a schedule manager 254, a user portal 256, and an authentication engine 258. Other microservices provided by system 250 are described and may be performed by any of these components as appropriate.

The schedule manager 254 is configured to receive, validate, and otherwise manage a specification of surgical implements (also referred to as a schedule). In various embodiments, the schedule manager includes an upload service that takes a CSV manifest with a link to a file and its associated metadata. Middleware may be provided to support the upload service. For example, the upload service may be accessed via an application programming interface (API), obtains a link where data can be uploaded to a secure bucket, and a queue may be managed as appropriate. The schedule manager provides a mechanism to securely store and access user reports and associated metadata. The schedule manager provides the client device 210 and 220 an endpoint to securely upload images, schedules, or other data and the user portal 256 the ability to search for them via metadata, allows census instruments to be stored and queried, and incorporates the centralized security of the auth service.

In various embodiments, the schedule manager 254 schedules import files. For example, weekly surgery information is pulled from an EHR. It has been observed that the schedule format may change on short notice. The schedule formats are accommodated by performing one or more transforms that can be embedded in a dynamic manifest for each facility, stored outside of code, e.g., stored in a database. The manifest provides a recipe to convert disparate schedule import file formats for different customers to a common format. This is beneficial compared with code, because the manifest can be altered on a faster cycle than a code release cycle.

The surgery information may be input via a UI and validated. For example, data may be expected in a format where a particular cell has a list or array of trays and peel packs related to a surgery or a row/multiple rows for a particular surgery that has a separate instrument tray or peel pack. The rows are condensed or collapsed into a particular surgery to recognize that a set of instruments or peel packs were used for that particular surgery. The tray names that are used in the medical records system (EMR/EHR) do not always map to the tray names that are available in the count sheets, which were provided separately and ingested separately.

In various embodiments, validation may be performed at one or more stages of data ingestion, conversion, or post-conversion. For example, pre-ingest validation that identifies/corrects common mistakes such as a missing column or rearranged columns. During-conversion validation finds incorrectly formatted dates or values. Post-conversion validation checks for logic errors such as whether the scheduled end time came before the scheduled start time. Validation errors may be displayed on a user interface with varying levels of granularity such as at the cell level, at the row level, or at the column level for a spreadsheet. Those records that may need adjustments may be surfaced prior to beginning the conversion stage.

Although not shown with respect to the image provider client device, a similar manager (image manager) may be provided and have similar functions.

The instrument classification engine 252 is configured to automatically generate a record of the utilized surgical implements depicted in the image and store the record in a data storage of surgical implement utilization data. As described herein, a received image may be manipulated in various ways. For example, the metadata of the image may be manipulated by performing a lookup based on metadata. For example, the engine looks up the OR and the facility and passes this information along for downstream processing. The information may be stored in an appropriate table such as a row in an image table, and a row in a tray analysis table.

The optimization engine 262 is configured to automatically analyze records stored in the data storage of surgical implement utilization data to identify one or more surgical implement preparation groupings optimizing one or more specified utilization metrics.

The authentication engine 258 is configured to provide authentication services for various microservices associated with system 250. The authentication service may be proprietary or may be provided by a third party service provider. The authentication service may provide user-based authentication for users accessing the portal 256, and/or device-based authentication.

The user portal 256 is configured to interface with users and provide information for display on devices such as client devices 210 and 220. In various embodiments, the user portal includes an internal portal for internal facing data collection review, and a user facing portal with a dashboard for users to obtain information or reports. Examples of a dashboard and reports are further described herein with respect to FIGS. 3A to 8D.

A particular user may be authenticated by an authentication engine 258 as further described herein. The user may be granted permissions to a specific hospital facility, where they can see reports for that facility, or for a system which is a collection of facilities, for example. Optimization reports may be indexed to be easily searched or otherwise accessed. The reports for which a user has permission to view and associated metadata are displayed to help the user easily find requested information. Filtering capabilities may also be provided.

A taxonomy may be defined to cover various types of reports so that reports are standardized to be comparable across different types of reports. Example reports include but are not limited to an optimizing report and a weekly update report. The optimizing report may be based on individual trays, procedures, surgeons, or the like. For example, the tray report may be made per surgeon, per procedure, per healthcare facility, per healthcare system etc. A weekly update report includes the status of client devices such as the image provider client device 210 and/or the schedule provider client device 220.

FIG. 3A shows an example of a user interface for instrument selection optimization. In this example, “ORion” refers to the disclosed instrument selection optimization system. Navigation within the dashboard may be facilitated by a menu, e.g., the menu at the top of the user interface showing that the current health system is “Acme Health,” and the dashboard is for “Loc 1” of the Acme Health system. One or more facilities and an associated number of captured trays are displayed in this example. Selecting element 302, which is a facility, causes the next user interface to be displayed.

FIG. 3B shows an example of a user interface for instrument selection optimization including tray analysis. In this example, each row corresponds to a tray. Associated tray information such as the number of images captured, percent usage, surgeons captured, and whether statistic significance has been reached may be displayed in corresponding columns as shown. Hovering over a particular tray causes additional details to be displayed in a pop-up window 304. The details include a list of surgeons and a corresponding number of images and percent usage. Selecting the tray “Major Hand Set” causes the next user interface to be displayed.

FIG. 3C shows an example of a user interface for instrument selection optimization including optimization information for a particular tray. In this example, information for the tray “Major Hand Set” is displayed. Savings in terms of OR & SPD Time, and dollar amount may be displayed as shown. The number of images captured and surgeons associated with the underlying data may be displayed. Selecting button 312 causes one or more reports to be generated, examples of which are shown in FIGS. 5A to 8D. Optimization and analysis may be based on the disclosed techniques such as the process of FIG. 1. In this example, optimization results corresponding to maximum savings, everything in the operating room (OR), and removing never used instruments are shown. Selecting button 314 causes a list of never used instruments to be displayed as shown in the next user interface. Selecting button 316 causes a list of suggestions to obtain maximum savings to be displayed as shown in FIG. 3E.

FIG. 3D shows an example of a user interface for instrument selection optimization including never used instruments. A list of never used items is shown in this example. This detail allows a user to view the specific implements that are part of a current tray (here, Major Hand Set) that are never used in any procedure by any surgeon. The user interface also includes a navigation panel 322 that allows the user to perform functions such as obtaining a PDF report, returning to a tray overview (FIG. 3C), selecting a new tray (FIG. 3B), or selecting a new facility (FIG. 3A).

FIG. 3E shows an example of a user interface for instrument selection optimization including an optimization for a particular tray. This optimization shows a suggested main tray that includes implements that are always used along with an accessory tray that includes implements that are less frequently used. A user is able to drill down to details by hovering over a particular instrument. In this example, a pop-up window 332 is displayed. The displayed information may be helpful for obtaining additional insight. For example, Dr. Cormorant may be contacted to ask why they so frequently use a forcep, Brown-Adson tissue 4⅞.

FIG. 4 shows an example of a user interface for an annotation engine. As described herein, an annotation engine may solicit input from a human annotator. This example user interface provides a user-friendly interface for viewing and/or annotating surgical implements. In this example, 12 options are shown, with the first six options being displayed along with a corresponding image of the implement.

In various embodiments, a corpus of data includes trays from users and a library with associated labels. This user interface displays and/or allows a human annotator to find a best match between an instrument label in the tray to a set in an existing image library. The matching may be based on cosine similarity and/or a scoring algorithm. For example, per unique symbol in a particular string, cosine similarity determines how many best matches there are. For example, for a “forceps bayonet smooth 7⅝,” best match may be based on “forceps,” “bayonet,” and “smooth.” There might not be an exact match for “7⅝.” That is the reason for rating the first option 402 more highly than the others. These determined top level matches may be recommended and displayed for a human annotator to confirm or pick secondary matches.

The following figures show examples of reports including optimization information determined by using the disclosed techniques. The content of the reports may be presented in various formats including in a dashboard such as the one described with respect to FIGS. 3A to 3E.

FIG. 5A shows an example of a report including a procedural playbook. This procedural playbook shows how to lay out instruments in a way that is personalized for a specific doctor (or more generally, medical practitioner). An image of an instrument is presented alongside the name to help easily identify the instrument. The playbook can be customized to each medical practitioner. For example, a nurse/technician can set up instruments according to the playbook. Like procedural economic insights, the procedural playbook also provides insights into how often specific instruments are used by a specific doctor.

FIG. 5B shows an example of a report including a procedural playbook. The information in FIG. 5B is like the one shown in FIG. 5A unless otherwise described. Unlike FIG. 5A, the information is presented without pictures and with the exact percent usage.

FIG. 6 shows an example of a report including a tray compliance roadmap. In various embodiments, the roadmap can be used to meet weight limits of trays and reduce workplace injuries.

FIG. 7 shows an example of a report including procedural economic insights. The insights may help establish best practices and generate surgeon report cards.

FIGS. 8A to 8D show examples of a report including service line optimization. In this example, an optimization is based on a generalization over different doctors. For example, systemwide cost containment and standardization can be improved.

FIG. 9 is a functional diagram illustrating a programmed computer system for instrument selection optimization in accordance with some embodiments. As will be apparent, other computer system architectures and configurations can be used to perform the described instrument selection optimization technique. Computer system 900, which includes various subsystems as described below, includes at least one microprocessor subsystem (also referred to as a processor or a central processing unit (CPU) 902). For example, processor 902 can be implemented by a single-chip processor or by multiple processors. In some embodiments, processor 902 is a general purpose digital processor that controls the operation of the computer system 900. In some embodiments, processor 902 also includes one or more coprocessors or special purpose processors (e.g., a graphics processor, a network processor, etc.). Using instructions retrieved from memory 910, processor 902 controls the reception and manipulation of input data received on an input device (e.g., image processing device 906, I/O device interface 904), and the output and display of data on output devices (e.g., display 918).

Processor 902 is coupled bi-directionally with memory 910, which can include, for example, one or more random access memories (RAM) and/or one or more read-only memories (ROM). As is well known in the art, memory 910 can be used as a general storage area, a temporary (e.g., scratch pad) memory, and/or a cache memory. Memory 910 can also be used to store input data and processed data, as well as to store programming instructions and data, in the form of data objects and text objects, in addition to other data and instructions for processes operating on processor 902. Also as is well known in the art, memory 910 typically includes basic operating instructions, program code, data, and objects used by the processor 902 to perform its functions (e.g., programmed instructions). For example, memory 910 can include any suitable computer readable storage media described below, depending on whether, for example, data access needs to be bi-directional or uni-directional. For example, processor 902 can also directly and very rapidly retrieve and store frequently needed data in a cache memory included in memory 910.

A removable mass storage device 912 provides additional data storage capacity for the computer system 900, and is optionally coupled either bi-directionally (read/write) or uni-directionally (read only) to processor 902. A fixed mass storage 920 can also, for example, provide additional data storage capacity. For example, storage devices 912 and/or 920 can include computer readable media such as magnetic tape, flash memory, PC-CARDS, portable mass storage devices such as hard drives (e.g., magnetic, optical, or solid state drives), holographic storage devices, and other storage devices. Mass storages 912 and/or 920 generally store additional programming instructions, data, and the like that typically are not in active use by the processor 902. It will be appreciated that the information retained within mass storages 912 and 920 can be incorporated, if needed, in standard fashion as part of memory 910 (e.g., RAM) as virtual memory.

In addition to providing processor 902 access to storage subsystems, bus 114 can be used to provide access to other subsystems and devices as well. As shown, these can include a display 918, a network interface 916, an input/output (I/O) device interface 904, an image processing device 906, as well as other subsystems and devices. For example, image processing device 906 can include a camera, a scanner, etc.; I/O device interface 904 can include a device interface for interacting with a touchscreen (e.g., a capacitive touch sensitive screen that supports gesture interpretation), a microphone, a sound card, a speaker, a keyboard, a pointing device (e.g., a mouse, a stylus, a human finger), a Global Positioning System (GPS) receiver, an accelerometer, and/or any other appropriate device interface for interacting with system 900. Multiple I/O device interfaces can be used in conjunction with computer system 900. The I/O device interface can include general and customized interfaces that allow the processor 902 to send and, more typically, receive data from other devices such as keyboards, pointing devices, microphones, touchscreens, transducer card readers, tape readers, voice or handwriting recognizers, biometrics readers, cameras, portable mass storage devices, and other computers.

The network interface 916 allows processor 902 to be coupled to another computer, computer network, or telecommunications network using a network connection as shown. For example, through the network interface 916, the processor 902 can receive information (e.g., data objects or program instructions) from another network, or output information to another network in the course of performing method/process steps. Information, often represented as a sequence of instructions to be executed on a processor, can be received from and outputted to another network. An interface card or similar device and appropriate software implemented by (e.g., executed/performed on) processor 902 can be used to connect the computer system 900 to an external network and transfer data according to standard protocols. For example, various process embodiments disclosed herein can be executed on processor 902, or can be performed across a network such as the Internet, intranet networks, or local area networks, in conjunction with a remote processor that shares a portion of the processing. Additional mass storage devices (not shown) can also be connected to processor 902 through network interface 916.

In addition, various embodiments disclosed herein further relate to computer storage products with a computer readable medium that includes program code for performing various computer-implemented operations. The computer readable medium includes any data storage device that can store data which can thereafter be read by a computer system. Examples of computer readable media include, but are not limited to: magnetic media such as disks and magnetic tape; optical media such as CD-ROM disks; magneto-optical media such as optical disks; and specially configured hardware devices such as application-specific integrated circuits (ASICs), programmable logic devices (PLDs), and ROM and RAM devices. Examples of program code include both machine code as produced, for example, by a compiler, or files containing higher level code (e.g., script) that can be executed using an interpreter.

The computer system shown in FIG. 9 is but an example of a computer system suitable for use with the various embodiments disclosed herein. Other computer systems suitable for such use can include additional or fewer subsystems. In some computer systems, subsystems can share components (e.g., for touchscreen-based devices such as smart phones, tablets, etc., I/O device interface 904 and display 918 share the touch sensitive screen component, which both detects user inputs and displays outputs to the user). In addition, bus 114 is illustrative of any interconnection scheme serving to link the subsystems. Other computer architectures having different configurations of subsystems can also be utilized.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A method, comprising:
receiving a specification of surgical implements available for use during a surgical operation;
receiving an image depicting at least a portion of the available surgical implements utilized during the surgical operation;
at least in part automatically generating a record of the utilized surgical implements depicted in the image;
storing the record in a data storage of surgical implement utilization data; and
automatically analyzing records stored in the data storage of surgical implement utilization data to identify one or more surgical implement preparation groupings optimizing one or more specified utilization metrics.

2. The method of claim 1, wherein the specification of the surgical implements available for use during the surgical operation is received from a first source and the image depicting the at least a portion of the available surgical implements utilized during the surgical operation is received from a second source different from the first source.

3. The method of claim 1, wherein the specification of the surgical implements available for use during the surgical operation includes an instrument count sheet.

4. The method of claim 1, wherein automatically generating the record of the utilized surgical implements depicted in the image includes annotating each of at least a subset of the utilized surgical implements based at least on a plurality of previously received images.

5. The method of claim 4, wherein a total number of the plurality of previously received images utilized for the annotation is based at least on a robustness analysis of the plurality of previously received images performed using a threshold indicating a reliability of the plurality of the previously received images.

6. The method of claim 4, wherein a total number of the plurality of previously received images utilized for the annotation is based at least on a coverage analysis of the plurality of previously received images performed using a threshold indicating a level of representation of the surgical operation with respect to a group of surgical operations.

7. The method of claim 1, wherein automatically generating the record of the utilized surgical implements depicted in the image includes identifying each of at least a subset of the utilized surgical implements based at least on a trained machine learning model.

8. The method of claim 1, wherein automatically generating the record of the utilized surgical implements depicted in the image includes identifying each of at least a subset of the utilized surgical implements without using the specification of the surgical implements available for use during the surgical operation.

9. The method of claim 1, wherein the record of the utilized surgical implements depicted in the image is based at least on the specification of the surgical implements available for use during the surgical operation.

10. The method of claim 1, wherein the one or more surgical implement preparation groupings are organized into one or more trays.

11. The method of claim 10, wherein the one or more trays includes a core tray including a first subset of the utilized surgical implements and an accessory tray including a second subset of the utilized surgical implements.

12. The method of claim 1, wherein automatically analyzing the records stored in the data storage of the surgical implement utilization data to identify the one or more surgical implement preparation groupings optimizing the one or more specified utilization metrics includes performing service-line analysis.

13. The method of claim 1, wherein automatically analyzing the records stored in the data storage of the surgical implement utilization data to identify the one or more surgical implement preparation groupings optimizing the one or more specified utilization metrics includes determining a co-usage pattern of a first one of the utilized surgical implements that is used with a second one of the utilized surgical implements at a frequency that meets a usage threshold.

14. The method of claim 1, further comprising outputting a report based at least on the automatic analysis of the records stored in the data storage of the surgical implement utilization data to identify the one or more surgical implement preparation groupings optimizing the one or more specified utilization metrics.

15. The method of claim 1, wherein the one or more specified utilization metrics includes at least one of: a corresponding usage rate for each instrument.

16. The method of claim 1, wherein automatically analyzing the records stored in the data storage of the surgical implement utilization data to identify the one or more surgical implement preparation groupings optimizing the one or more specified utilization metrics includes performing a robustness analysis to determine a reliability of the identified the one or more surgical implement preparation groupings.

17. The method of claim 1, wherein automatically analyzing the records stored in the data storage of the surgical implement utilization data to identify the one or more surgical implement preparation groupings optimizing the one or more specified utilization metrics includes performing a coverage analysis to determine a representation level of a characteristic associated with the surgical operation.

18. The method of claim 17, wherein automatically analyzing the records stored in the data storage of the surgical implement utilization data to identify the one or more surgical implement preparation groupings optimizing the one or more specified utilization metrics includes performing a coverage analysis to determine a representation level of a characteristic associated with the surgical operation based at least on the specification of the surgical implements available for use during the surgical operation.

19. A system, comprising:
a processor configured to:
receive a specification of surgical implements available for use during a surgical operation;
receive an image depicting at least a portion of the available surgical implements utilized during the surgical operation;
at least in part automatically generate a record of the utilized surgical implements depicted in the image;
store the record in a data storage of surgical implement utilization data; and
automatically analyze records stored in the data storage of surgical implement utilization data to identify one or more surgical implement preparation groupings optimizing one or more specified utilization metrics; and
a memory coupled to the processor and configured to provide the processor with instructions.

20. A computer program product embodied in a non-transitory computer readable medium and comprising computer instructions for:
receiving a specification of surgical implements available for use during a surgical operation;
receiving an image depicting at least a portion of the available surgical implements utilized during the surgical operation;
at least in part automatically generating a record of the utilized surgical implements depicted in the image;
storing the record in a data storage of surgical implement utilization data; and
automatically analyzing records stored in the data storage of surgical implement utilization data to identify one or more surgical implement preparation groupings optimizing one or more specified utilization metrics.
